(19) United States
(12) Patent Application Publication
(10) Pub. No.: US 2025/0388556 A1
(43) Pub. Date: Dec. 25, 2025

(54) PROCESS FOR PREPARING EPICHLOROHYDRIN BY DIRECTLY OXIDIZING CHLOROPROPENE BY USING LIQUID-SOLID CIRCULATING FLUIDIZED BED REACTOR

(71) Applicant: DALIAN INSTITUTE OF CHEMICAL PHYSICS, CHINESE ACADEMY OF SCIENCES, Dalian (CN)

(72) Inventors: Jiahui HUANG, Dalian (CN); Yingchun LIU, Dalian (CN); Xin SU, Dalian (CN); Guse HE, Dalian (CN); Hengyun ZHANG, Dalian (CN); Peng HE, Dalian (CN); Changyun WANG, Dalian (CN); Xinju LI, Dalian (CN); Yuhua JIA, Dalian (CN); Huayun LONG, Dalian (CN)

(21) Appl. No.: 19/314,050

(22) Filed: Aug. 29, 2025

Related U.S. Application Data
(63) Continuation-in-part of application No. PCT/CN2023/086469, filed on Apr. 6, 2023.

(30) Foreign Application Priority Data
Dec. 1, 2022 (CN) 202211539106.X

(51) Int. Cl. C07D 301/12; B01J 35/40; B01J 35/51; B01J 38/48

(52) U.S. Cl. CPC C07D 301/12; B01J 35/40; B01J 35/51; B01J 38/48

(57) ABSTRACT

A process for preparing epichlorohydrin is by directly oxidizing chloropropene with hydrogen peroxide using a liquid-solid circulating fluidized bed reactor. The liquid-solid circulating fluidized bed reactor has a reactor, a liquid-solid separator, a liquid extractor, a spent material inclined tube, a regenerator, a catalyst bin, a regeneration inclined tube, etc. In the process, the liquid-solid circulating fluidized bed reactor is used to replace a traditional stirred tank reactor, such that the heat and mass transfer rate between liquid and solid phases is enhanced in the reactor, the back mixing degree in the reactor is reduced, the reaction rate is greatly increased, the reaction time is shortened, side reactions are inhibited, and the effective utilization rate of hydrogen peroxide and the selectivity of epichlorohydrin are increased.

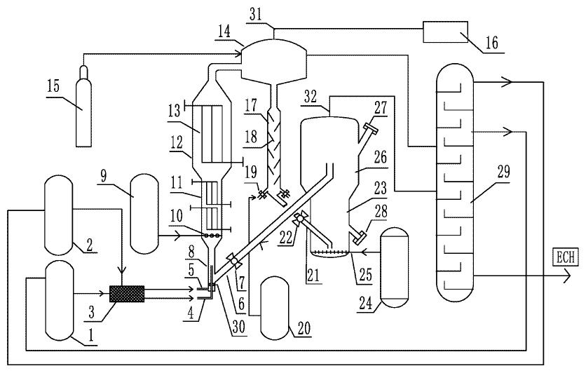

### TECHNICAL FIELD

The present disclosure belongs to the technical field of epichlorohydrin production, in particular to a process for preparing epichlorohydrin by directly oxidizing chloropropene with hydrogen peroxide by using a liquid-solid circulating fluidized bed reactor.

### BACKGROUND ART

Epichlorohydrin (abbreviated as ECH) is an important organic chemical raw material and fine chemical product, widely used in productions of epoxy resins, chlorohydrin rubber, synthetic glycerol and the like. In addition, epichlorohydrin is also widely used to produce in manufacturing products such as of ion exchange resins, adhesives, surfactants, architectural coatings and pharmaceuticals. Currently, there are three main production processes for epichlorohydrin: propylene high-temperature chlorination method, propylene acetate method and glycerine method.

The propylene high-temperature chlorination method primarily uses propylene, chlorine gas and lime milk as raw materials to produce epichlorohydrin, involving three steps of high-temperature chlorination of propylene, hypo chlorination of chloropropene and saponification of dichloropropanol. The process is the most classic epichlorohydrin synthesis process, with a history of over 70 years. However, the process has a low yield, with a utilization rate of chlorine atoms of only about 25%. In addition, a large amount of chlorine-containing wastewater and calcium chloride waste residues are generated during the reaction process, causing severe environmental pollution. Approximately 40 tons of chlorine-containing wastewater produced per ton of epichlorohydrin manufactured.

The propylene acetate method uses propylene, oxygen, acetic acid, chlorine gas and lime milk as raw materials, involving oxidation reaction of propylene with acetic acid to produce propylene acetate, hydrolyzation of the propylene acetate to obtain allyl alcohol, chlorination of the propylene alcohol to produce dichloropropanol, and saponification of the dichloropropanol to finally produce epichlorohydrin. However, this process also generates a large amount of wastewater and waste residues, and the reaction route is long with high investment. Industrial production using this process has been ceased both at domestically and internationally.

The glycerine method primarily uses glycerol, hydrogen chloride and lime milk as raw materials to produce epichlorohydrin, involving two steps of glycerol chlorination and dichloropropanol saponification. The amount of wastewater generated in this process is only one-tenth of that produced by the propylene high-temperature chlorination method. However, the price fluctuation of glycerol fluctuates significantly, and the operation economy of the device is poor.

The direct oxidation of chloropropene to produce epichlorohydrin has wastewater discharge of only about 5% of those from the propylene high-temperature chlorination method, and has an atomic utilization rate as high as 84% with almost no waste residue generated, which truly realizes green and clean production of epichlorohydrin. The direct epoxidation process of chloropropene using hydrogen peroxide as an oxidant has become a research hotspot. This process uses TS-1 titanium silicalite molecular sieve as a catalyst to realize the direct epoxidation reaction of chloropropene with hydrogen peroxide in methanol solvent to produce epichlorohydrin. A continuous stirred-tank reactor is often used in laboratories for this reaction process. However, since sever backmixing in the experiment process, hydrogen peroxide cannot be completely converted, posing certain safety risks for subsequent processes. Moreover, the residence time of reactants and products in the reactor is relatively long, leading to rapid catalyst deactivation, increased side reaction, and lower effective utilization of hydrogen peroxide and selectivity for epichlorohydrin.

### SUMMARY

In view of the problems in the prior art, an objective of the present disclosure is to provide a process for preparing epichlorohydrin by directly oxidizing chloropropene by using a liquid-solid circulating fluidized bed reactor. The process of the present disclosure achieves a hydrogen peroxide conversion rate of up to 99.9%, an effective hydrogen peroxide utilization rate of up to 96.0%, and an epichlorohydrin selectivity of up to 99.0%. Compared with the continuous stirred-tank reactor, the process of the present disclosure significantly shortens reaction time and greatly improves reaction efficiency.

The objective of the present disclosure is achieved by the following technical solutions:

The present disclosure provides a process for preparing epichlorohydrin by directly oxidizing chloropropene by using a liquid-solid circulating fluidized bed reactor. The liquid-solid circulating fluidized bed reactor comprises a reactor inlet 8, a first reactor 11, a second reactor 12, a liquid-solid separator 14, a liquid extractor 17, a spent material inclined tube 21, a regenerator 23, a catalyst bin 26, and a regeneration inclined tube 6 sequentially connected in series. An end of the regeneration inclined tube 6 is connected to the reactor inlet 8, and the reactor inlet 8 is provided with an inlet structure at its bottom, with its sidewall connected to the regeneration inclined tube 6 and its top connected to the first reactor 11. An upper end of the first reactor 11 is disposed with the second reactor 12, and a reaction heat exchange system 13 is disposed in each of the two reactors. A tail end of the second reactor 12 is connected to the liquid-solid separator 14, a top end of the liquid-solid separator 14 is provided with a gas-phase outlet 31 connected to a tail gas treatment system 16 via a pipeline, a side wall of the liquid-solid separator 14 is connected to a solvent circulation and separation system 29 via a pipeline, a lower end of the liquid-solid separator 14 is connected to a top of the liquid extractor 17, and the liquid extractor 17 is provided with an internal component 18 inside and a cleaning liquid inlet 19 at its bottom. A bottom of the liquid extractor 17 is connected to the spent material inclined tube 21, which is connected to a bottom of the regenerator 23. The bottom of the regenerator is provided with a regeneration liquid distributor 25, and an upper part of the regenerator 23 is connected to the catalyst bin 26, the regeneration inclined tube 6 with an upward opening is disposed on an axis of the catalyst bin, and a top of the regenerator 23 is provided with a regeneration liquid outlet 32. A middle of the regeneration inclined tube 6 is provided with a regenerant control valve 7. A solvent outlet of the solvent circulation and separation system 29 is connected to the a solvent storage tank 1 via a pipeline, a chloropropene outlet of that is connected to a chloropropene storage tank 2 via a pipeline, and the prepared epichlorohydrin is separated from a bottom of the solvent circulation and separation system 29.

The liquid-solid circulating fluidized bed reactor is used to directly oxidize chloropropene with hydrogen peroxide to prepare epichlorohydrin, and the preparation process comprises the following steps of:
(1) Mixing reactants of chloropropene, hydrogen peroxide, a solvent and a catalyst at the reactor inlet 8, and then sequentially entering the first reactor 11 and the second reactor 12 for epoxidation reaction to obtain a liquid-solid mixture containing epichlorohydrin;
(2) Introducing the liquid-solid mixture after the reaction into the liquid-solid separator 14 for liquid-solid separation; introducing a separated liquid-phase product into the solvent circulation and separation system 29, where a separated solvent is recycled back to the solvent storage tank 1, a separated unreacted chloropropene is recycled back to the chloropropene storage tank 2, and the epichlorohydrin after purification is output as a product; and introducing separated catalyst particles into the liquid extractor 17; and introducing nitrogen from a nitrogen storage tank 15 into a top of the liquid-solid separator 14 to dilute oxygen generated by a self-decomposition of hydrogen peroxide, ensuring safe operation of the separator, and introducing a diluted gas-phase into the tail gas treatment system 16 before being released into the atmosphere;
(3) Washing the catalyst entering the liquid extractor 17 to remove residual reaction products in catalyst gaps under an action of a cleaning liquid, the cleaning liquid countercurrently flowing into the liquid-solid separator 14, and introducing the cleaned catalyst to the bottom of the regenerator 23 along the spent material inclined tube 21, where the liquid extractor 17 is provided with an internal component 18 to increase the liquid-solid contact efficiency and improve the cleaning efficiency while form a granular bed in the spent material inclined tube 21 to prevent backflow of the regenerated liquid from the regenerator 23 into the liquid-solid separator 14; and
(4) Physically or chemically regenerating the catalyst entering into the regenerator 23 under an action of a regeneration liquid, the regenerated catalyst entering the catalyst bin 26 followed by entering the reactor inlet 8 through the regeneration inclined tube 6 to participate in the reaction again to realizes cyclic regeneration of the catalyst, and the regeneration liquid entering the solvent circulation and separation system 29 from the regeneration liquid outlet 32 at a top of the catalyst bin 23.

Further, the bottom of the reactor inlet 8 is configured as a Venturi-type inlet structure to enhance turbulence and mixing between the liquid and solid phases, improving liquid-solid contact efficiency.

Further, the reactor inlet 8 is provided with a main liquid inlet 4 at the bottom axis of the reactor inlet 8 and an auxiliary liquid inlet 5. The liquid entering the auxiliary liquid inlet 5 is uniformly distributed into the reactor through an auxiliary liquid distributor 30. Both the main liquid inlet 4 and the auxiliary liquid inlet 5 are connected to a mixer 3, and the mixer 3 is connected to the solvent storage tank 1 and the raw material chloropropene storage tank 2 via pipelines, respectively.

Further, a diameter of the second reactor 12 is greater than that of the first reactor 11. A flow velocity of reactants in the first reactor 11 is high, enabling intense reaction and rapid removal of a large amount of heat. The reaction in the second reactor 12 is relatively moderate, and a residence time of the reactants is prolonged by increasing the inner diameter of the reactor to reduce flow velocity.

Further, a liquid extractor 17 is disposed at the bottom of the liquid-solid separator 14 to extract reaction products from the surface of catalyst, thereby improving the yield of epichlorohydrin and inhibiting side reactions. The liquid extractor 17 is provided with an internal component 18, which is at least one selected from herringbone-type, grid-type, disc-ring-type, and packing.

Further, the catalyst after the reaction is physically or chemically regenerated in the regenerator 23, and a superficial liquid velocity of the regeneration liquid in the regenerator 23 is 1-100 times the minimum fluidization velocity of the catalyst particles.

Further, an upper part of the sidewall of the regenerator 23 is provided with a catalyst inlet 27, and a lower part of the sidewall of the regenerator 23 is provided with a catalyst outlet 28.

Further, the catalyst used in the reaction system is a microspherical TS-1 catalyst, with a particle size distribution of the catalyst is 0.03-6 mm, and a particle density is of 500-8000 kg/m³.

Further, the reaction solvent in the reactor, the cleaning liquid in the liquid extractor 17, and the physical regeneration liquid in the regenerator 23 are selected from methanol, ethanol, acetone, acetonitrile, chloroform, 1,4-dioxane, isopropanol, and tert-butyl alcohol, and mixtures thereof.

Further, a superficial liquid velocity of the mixture in the first reactor is 1-6000 m/h, a molar ratio of the hydrogen peroxide to the chloropropene is 1:1-1:10, and a molar ratio of the hydrogen peroxide to the solvent is 1:2-1:15. A concentration of the hydrogen peroxide is 5-70%.

Further, a reaction temperature is controlled at 0-100° C., and a pressure inside the reactor is controlled at 0.01-5 MPa.

Further, an effective height of the first reactor is 5-60 m, an effective height of the second reactor is 0-30 m, a ratio of the inner diameter of the first reactor to that of the second reactor is 1:1-1:5, and a liquid-phase residence time is 3-300 min.

Compared to the prior art, the present disclosure has the following beneficial effects:

The present disclosure uses a liquid-solid circulating fluidized bed reactor to realize the process for preparing epichlorohydrin by oxidizing chloropropene, with a hydrogen peroxide conversion rate of up to 99.9%, an effective hydrogen peroxide utilization rate of up to 96.0%, and an epichlorohydrin selectivity of up to 99.0%. Compared with the continuous stirred-tank reactor, the process of the present disclosure significantly shortens reaction time and greatly improves reaction efficiency.

### BRIEF DESCRIPTION OF THE DRAWINGS

In order to more clearly describe embodiments of the present disclosure, a brief d to the accompanying drawings required for the description of the embodiments will be provided below.

The FIGURE is a flowchart of a process for preparing epichlorohydrin by directly oxidizing chloropropene by using a liquid-solid circulating fluidized bed reactor of the present disclosure, wherein, 1-solvent storage tank, 2-raw material chloropropene storage tank, 3-mixer, 4-main liquid inlet, 5-auxiliary liquid inlet, 6-regeneration inclined tube, 7-regenerant control valve, 8-reactor inlet, 9-hydrogen peroxide strange tank, 10-hydrogen peroxide inlet, 11-first reactor, 12-second reactor, 13-reaction heat exchange system, 14-liquid-solid separator, 15-nitrogen storage tank, 16-tail gas treatment system, 17-extractor, 18-internal component of liquid extractor, 19-cleaning liquid inlet, 20-cleaning liquid storage tank, 21-spent material inclined tube, 22-spent material solvent control valve, 23-regenerator, 24-regeneration liquid storage tank, 25-regeneration liquid distributor, 26-catalyst bin, 27-catalyst inlet, 28-catalyst outlet, 29-solvent circulation and separation system, 30-auxiliary liquid distributor, 31-gas-phase outlet, 32-regeneration liquid outlet.

### DETAILED DESCRIPTION OF THE EMBODIMENTS

The following is a further description of the present disclosure in conjunction with embodiments, and the embodiments should be regarded as demonstrative and non-limiting. Obviously, the described embodiments are only some embodiments of the present disclosure. Other similar embodiments obtained by those ordinary skilled in the art without creative labor should fall within the protection scope of the present disclosure.

### EMBODIMENT

This embodiment provides a process for preparing epichlorohydrin by directly oxidizing chloropropene by using a liquid-solid circulating fluidized bed reactor, which uses a liquid-solid circulating fluidized bed reactor to produce epichlorohydrin by directly oxidizing chloropropene with hydrogen peroxide. A flowchart of the process is shown in the FIGURE. The liquid-solid circulating fluidized bed reactor comprises a reactor inlet 8, a first reactor 11, a second reactor 12, a liquid-solid separator 14, a liquid extractor 17, a spent material inclined tube 21, a regenerator 23, a catalyst bin 26, and a regeneration inclined tube 6 sequentially connected in series. An end of the regeneration inclined tube 6 is connected to the reactor inlet 8, and the reactor inlet 8 is provided with an inlet structure at its bottom, with its sidewall connected to the regeneration inclined tube 6 and its top connected to the first reactor 11. An upper end of the first reactor 11 is disposed with the second reactor 12, and a reaction heat exchange system 13 is disposed in each of the two reactors. A tail end of the second reactor 12 is connected to the liquid-solid separator 14, a top end of the liquid-solid separator 14 is provided with a gas-phase outlet 31 connected to a tail gas treatment system 16 via a pipeline, a side wall of the liquid-solid separator 14 is connected to a solvent circulation and separation system 29 via a pipeline, a lower end of the liquid-solid separator 14 is connected to a top of the liquid extractor 17, and the liquid extractor 17 is provided with an internal component 18 inside and a cleaning liquid inlet 19 at its bottom. A bottom of the liquid extractor 17 is connected to the spent material inclined tube 21, which is connected to a bottom of the regenerator 23. The bottom of the regenerator is provided with a regeneration liquid distributor 25, and an upper part of the regenerator 23 is connected to the catalyst bin 26, the regeneration inclined tube 6 with an upward opening is disposed at a middle of the catalyst bin, and a top of the regenerator 23 is provided with a regeneration liquid outlet 32. An end of the regeneration inclined pipe 6 is directly connected with a sidewall of the reactor inlet 8, and a middle of the regeneration inclined tube 6 is provided with a regenerant control valve 7. A solvent outlet of the solvent circulation and separation system 29 is connected to the a solvent storage tank 1 via a pipeline, a chloropropene outlet of that is connected to a chloropropene storage tank 2 via a pipeline, and the prepared epichlorohydrin is separated from a bottom of the solvent circulation and separation system 29. A top of the catalyst bin is connected to the solvent circulation and separation system 29 through the regeneration liquid outlet 32. The reaction system can be used in the process for producing epichlorohydrin by directly oxidizing chloropropene with hydrogen peroxide.

The process mainly includes the following steps:
(1) Reactants of chloropropene, hydrogen peroxide, a solvent and a catalyst are mixed at the reactor inlet 8, which sequentially enter the first reactor 11 and the second reactor 12 for epoxidation reaction to obtain a liquid-solid mixture containing epichlorohydrin;
(2) The liquid-solid mixture after the reaction enters the liquid-solid separator 14 for liquid-solid separation; the separated liquid-phase product is introduced into the solvent circulation and separation system 29, where the separated solvent is recycled back to the solvent storage tank 1, the unreacted chloropropene is recycled back to the chloropropene storage tank 2, and the epichlorohydrin after purification is output as a product; the separated catalyst particles is introduced into the liquid extractor 17; in addition, nitrogen is introduced into the top of the liquid-solid separator 14 from a nitrogen storage tank 15 to dilute the oxygen generated by a self-decomposition of hydrogen peroxide, ensuring safe operation of the separator; and the diluted gas-phase is introduced into a tail gas treatment system 16 before being released into the atmosphere;
(3) The catalyst entering the liquid extractor 17 is washed to remove residual reaction products in catalyst gaps under an action of a cleaning liquid, the cleaning liquid countercurrently flows into the liquid-solid separator 14, and the cleaned catalyst is introduced to the bottom of the regenerator 23 along the spent material inclined tube 21; the liquid extractor 17 is provided with an internal component 18 to increase the liquid-solid contact efficiency and improve the cleaning efficiency while form a granular bed in the spent material inclined tube 21 to prevent backflow of the regenerated liquid from the regenerator 23 into the liquid-solid separator 14; and
(4) The catalyst entering into the regenerator 23 is regenerated physically or chemically under an action of the regeneration liquid, the regenerated catalyst is introduced into the catalyst bin 26 followed by entering the reactor inlet through the regeneration inclined tube 6 to participate in the reaction again to realize cyclic regeneration of the catalyst, and the regeneration liquid is introduced into the solvent circulation and separation system 29 from the regeneration liquid outlet 32 at a top of the catalyst bin 23.

A specific process is as follows:
Methanol from the solvent storage tank 1 and chloropropene from the chloropropene storage tank 2 are fully and uniformly mixed in the mixer 3, the obtained mixture is introduced into the bottom of the reactor through the main liquid inlet 4 at a certain flow rate. Meanwhile, a portion of the mixture of chloropropene and methanol is uniformly introduced into the reactor through the auxiliary liquid inlet 5 under the action of the auxiliary liquid distributor 30, and is mixed with the regenerant from the regeneration inclined tube 6 and moved axially upward. The circulation rate of the catalyst entering the reactor—i.e., the catalyst concentration in the reactor—is controlled by the regenerant control valve 7, with a preferred mass fraction of the catalyst in the reactor of 0.5 wt %. A 50 wt % hydrogen peroxide from the hydrogen peroxide storage tank 9 is introduced into the upper part of the reactor inlet 8 through the hydrogen peroxide inlet 10, and is fully mixed with the methanol, chloropropene and catalyst under a jet action at the reactor inlet 8, followed by moving axially upward along the first reactor and undergoing an epoxidation reaction. In one embodiment, the molar ratio of the methanol, chloropropene and hydrogen peroxide in the reactor was 9:4:1. The reaction temperature was controlled to be 40° C. by using the reaction heat exchange system 13, the pressure of the reaction system was controlled to be 0.1 Mpa, the total effective height of the reactor was 6 m, the ratio of the inner diameter of the first reactor to that of the second reactor was 1:1, and the liquid residence time was 10 min.

As the reaction proceeds, the hydrogen peroxide is basically completely consumed, and the catalyst and liquid-phase products (mainly including methanol, unreacted chloropropene, produced epichlorohydrin, and water) after reaction are introduced into the liquid-solid separator 14. The liquid-phase products after liquid-solid separation are introduced into the solvent circulation and separation system 29. A certain amount of nitrogen is introduced into the top of the liquid-solid separator 14 to dilute oxygen generated by self-decomposition of the hydrogen peroxide in the reaction process, reducing the oxygen concentration in the gas-phase space and improving operation safety of the separator. The diluted gas is finally introduced into the tail gas treatment system 16. The separated catalyst settles to the top of the liquid extractor 17 and moves downward along the liquid extractor 17, which fully contact with the methanol countercurrently introduced by the cleaning liquid inlet 19 at the bottom of the liquid extractor 17 under the action of the herringbone baffles of the internal component 18 in the liquid extractor to wash away residual reaction products trapped between and on the surface of the catalyst particles. As the cleaning liquid methanol flows upward, the residual products washed from the catalyst finally enters the solvent circulation and separation system 29 through the liquid-solid separator 14.

The catalyst from the liquid extractor is introduced into the bottom of the regenerator 23 through the spent material inclined tube 21. The height of the catalyst bed in the liquid extractor can be effectively regulated through the spent material control valve, creating a seal state to prevent the products in the liquid-solid separator 14 from entering the regenerator. The catalyst entering the regenerator is further washed by the regeneration liquid methanol introduced by the regeneration liquid distributor 25 to remove residual products and oligomers on the surface and in the pores of the catalyst, thereby regenerating the catalyst. During regeneration, the catalyst slowly rises to the catalyst bin 26. When the catalyst reaches a position above the inlet of the regeneration inclined pipe 6, it is introduced into the bottom of the reactor inlet 8 through the regeneration inclined tube 6 and participates in the reaction again, forming a catalyst cyclic operation of reaction-regeneration-reaction. The regeneration liquid is introduced into the solvent recovery and separation system 29 through the regeneration liquid outlet 32 at the top of the catalyst bin. The methanol and chloropropene in the separation system are recycled back again to the solvent storage tank 1 and the chloropropene storage tank 2 for recycling after separation and refinement.

According to the operation procedure of the above embodiment, the process for preparing epichlorohydrin by directly oxidizing chloropropene by using a liquid-solid circulating fluidized bed reactor realizes a hydrogen peroxide conversion rate of up to 99.9%, an effective hydrogen peroxide utilization rate of up to 96.0%, and an epichlorohydrin selectivity of up to 99.0%. Compared with the continuous stirred-tank reactor, the process of the present disclosure significantly shortens reaction time and greatly improves reaction efficiency. The results are shown in Table 1.

TABLE 1
Comparison of oxidizing chloropropene with
hydrogen peroxide using different reactors

| | Liquid-solid | Continuous |
|---|---|---|
| | circulating | stirred-tank |
| | fluidized bed | reactor |
| Pressure, MPa | 0.1 | 0.1 |
| Temperature, ° C. | 40 | 40 |
| Reaction time, min | 10 | 240 |
| Hydrogen peroxide | 99.9 | 92.4 |
| conversion rate, % | | |
| Effective hydrogen | 96.0 | 91.6 |
| peroxide utilization rate, % | | |
| Epichlorohydrin | 99.0 | 96.9 |
| selectivity, % | | |

At last, it should be noted that the above various embodiments are merely intended to illustrate the technical solution of the present disclosure and not to limit the same; although the present disclosure has been described in detail with reference to the foregoing embodiments, it should be understood by those ordinary skilled in the art that the technical solutions described in the foregoing embodiments can be modified or equivalents can be substituted for some or all of the technical features thereof; and the modification or substitution does not make the essence of the corresponding technical solution deviate from the scope of the technical solution of each embodiment of the present disclosure.

1. A process for preparing epichlorohydrin by directly oxidizing chloropropene by using a liquid-solid circulating fluidized bed reactor, wherein the liquid-solid circulating fluidized bed reactor comprises a reactor inlet, a first reactor, a second reactor, a liquid-solid separator, a liquid extractor, a spent material inclined tube, a regenerator, a catalyst bin and a regeneration inclined tube sequentially connected in series, wherein an end of the regeneration inclined tube is connected to the reactor inlet, the reactor inlet is provided with an inlet structure at its bottom, with its sidewall connected to the regeneration inclined tube and its top connected to the first reactor; an upper end of the first reactor is disposed with the second reactor, a reaction heat exchange system is disposed in each of the two reactors; a tail end of the second reactor is connected to the liquid-solid separator, a top end of the liquid-solid separator is provided with a gas-phase outlet connected to a tail gas treatment system via a pipeline, a side wall of the liquid-solid separator is connected to a solvent circulation and separation system via a pipeline, a lower end of the liquid-solid separator is connected to a top of the liquid extractor, the liquid extractor is provided with an internal component inside and a cleaning liquid inlet at its bottom; a bottom of the liquid extractor is connected to the spent material inclined tube, which is connected to a bottom of the regenerator; the bottom of the regenerator is provided with a regeneration liquid distributor, an upper part of the regenerator is connected to the catalyst bin, the regeneration inclined tube with an upward opening is disposed on an axis of the catalyst bin, and a top of the regenerator is provided with a regeneration liquid outlet; a middle of the regeneration inclined tube is provided with a regenerant control valve; a solvent outlet of the solvent circulation and separation system is connected to the a solvent storage tank via a pipeline, a chloropropene outlet of that is connected to a chloropropene storage tank via a pipeline, and the prepared epichlorohydrin is separated from a bottom of the solvent circulation and separation system; said liquid-solid circulating fluidized bed reactor is used to directly oxidize chloropropene with hydrogen peroxide to prepare epichlorohydrin, and the preparation process comprises the following steps of:
(1) mixing reactants of chloropropene, hydrogen peroxide, a solvent and a catalyst at the reactor inlet, and then sequentially entering the first reactor and the second reactor for epoxidation reaction to obtain a liquid-solid mixture containing epichlorohydrin;
(2) introducing the liquid-solid mixture after the reaction into the liquid-solid separator for liquid-solid separation; and introducing a separated liquid-phase product into the solvent circulation and separation system, wherein a separated solvent is recycled back to the solvent storage tank, a separated unreacted chloropropene is recycled back to the chloropropene storage tank, and the epichlorohydrin after purification is output as a product; and introducing separated catalyst particles into the liquid extractor; and introducing nitrogen from a nitrogen storage tank into a top of the liquid-solid separator to dilute oxygen generated by a self-decomposition of hydrogen peroxide, ensuring safe operation of the separator, and introducing a diluted gas-phase into the tail gas treatment system before being released into the atmosphere;
(3) washing the catalyst entering the liquid extractor to remove residual reaction products in catalyst gaps under an action of a cleaning liquid, the cleaning liquid countercurrently flowing into the liquid-solid separator, and introducing the cleaned catalyst to the bottom of the regenerator along the spent material inclined tube; and
(4) physically or chemically regenerating the catalyst entering into the regenerator under an action of a regeneration liquid, the regenerated catalyst entering the catalyst bin followed by entering the reactor inlet through the regeneration inclined tube to participate in the reaction again to realize cyclic regeneration of the catalyst, and the regeneration liquid entering the solvent circulation and separation system from the regeneration liquid outlet at a top of the catalyst bin.

2. The process according to claim 1, wherein the bottom of the reactor inlet is configured as a Venturi-type inlet structure to enhance turbulence and mixing between the liquid and solid phases, improving liquid-solid contact efficiency.

3. The process according to claim 1, wherein a diameter of the second reactor is greater than that of the first reactor; a flow velocity of reactants in the first reactor is high, enabling intense reaction and rapid removal of a large amount of heat; and the reaction in the second reactor is relatively moderate, and a residence time of the reactant is prolonged by increasing the inner diameter of the reactor to reduce flow velocity.

4. The process according to claim 1, wherein the internal component disposed in the liquid extractor is at least one selected from herringbone-type, grid-type, disc-ring-type, and packing.

5. The process according to claim 1, wherein the catalyst after the reaction is physically or chemically regenerated in the regenerator, and a superficial liquid velocity of the regeneration liquid in the regenerator is 1-100 times the minimum fluidization velocity of the catalyst particles.

6. The process according to claim 1, wherein an upper part of a sidewall of the regenerator is provided with a catalyst inlet, and a lower part of the sidewall of the regenerator is provided with a catalyst outlet; and the catalyst used in the reaction system is a microspherical TS-1 catalyst with a particle size distribution of 0.03-6 mm and a particle density of 500-8000 kg/m³.

7. The process according to claim 1, wherein a reaction solvent in the reactor, the cleaning liquid in the liquid extractor, and the physical regeneration liquid in the regenerator are selected from methanol, ethanol, acetone, acetonitrile, chloroform, 1,4-dioxane, isopropanol, tert-butyl alcohol, and mixtures thereof.

8. The process according to claim 1, wherein a superficial liquid velocity of the mixture in the first reactor is 1-6000 m/h, a molar ratio of the hydrogen peroxide to the chloropropene is 1:1-1:10, a molar ratio of the hydrogen peroxide to the solvent is 1:2-1:15, and a concentration of the hydrogen peroxide is 5-70%.

9. The process according to claim 1, wherein a reaction temperature is controlled at 0-100° C., and a pressure inside the reactor is controlled at 0.01-5 MPa.

10. The process according to claim 1, wherein an effective height of the first reactor is 5-60 m, an effective height of the second reactor is 0-30 m, a ratio of the inner diameter of the first reactor to the inner diameter of the second reactor is 1:1-1:5, and a liquid-phase residence time is 3-300 min.
